(19) United States
(12) Patent Application Publication
(10) Pub. No.: US 2004/0234964 A1
(43) Pub. Date: Nov. 25, 2004

(54) Methods for reducing background on polynucleotide arrays

(75) Inventors: Kyle B. Cole, Stanford, CA (US); Vivi Truong, Mountain View, CA (US); Glenn McGall, San Jose, CA (US); Sumathi Venkatapathy, San Jose, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/441,864

(22) Filed: May 19, 2003

(51) Int. Cl. C12Q 1/68

(52) U.S. Cl. 435/6; 525/54.2

(57) ABSTRACT

Methods are provided for reducing background signal associated with hybridization of nucleic acid arrays to nucleic acid samples, the method comprising hybridizing the array to the sample in the presence of a poly-anionic polymer (PAP). Background associated with hybridization of arrays to samples interferes with signal generated by specific binding to the probe array un-desirably lowers the signal to noise ratio (SNR) and generates an overall loss of sensitivity for nucleic acid arrays.

Methods are also provided for hybridizing a nucleic acid sample to a nucleic acid array comprising incubating said sample with said array in the presence of a PAP. Hybridization buffers are also provided comprising a PAP.

### FIELD OF THE INVENTION

The present invention relates generally to the field of nucleic acid arrays. More specifically, the present invention relates to methods for reducing background and increasing readability of nucleic acid arrays.

### BACKGROUND OF THE INVENTION

Many biological functions are accomplished by altering the transcriptional profile of various genes. For example, fundamental biological processes such as cell cycle progression, cell differentiation and cell death, are often characterized by variations in gene expression levels.

Nucleic acid hybridizations are commonly used in biochemical research and diagnostic assays. Generally a single stranded nucleic acid is hybridized to labeled nucleic acid probe, and resulting nucleic acid duplexes are detected. Radioactive and non-radioactive labels have been used. Methods also have been developed to amplify the signal that is detected. Avidin-biotin systems have been developed for use in a variety of detection assays. Methods for the detection and labeling of nucleic acids in biotin systems are described, for example, in “Nonradioactive Labeling and Detection Systems”, C. Kessler, Ed., Springer-Verlag, New York, 1992, pp. 70-99; and in “Methods in Nonradioactive Detection,”, G. Howard, Ed., Appleton and Lange, Norwalk, Conn. 1993, pp. 11-27 and 137-150.

### SUMMARY OF THE INVENTION

Methods are provided for reducing background in nucleic acid arrays, the background associated with hybridization of the array with a nucleic acid sample, the method comprising hybridizing the array with the sample in the presence of a poly-anionic polymer (PAP). Sources of non-target signal that may cause background can include impurities, such as cell debris and salts in the nucleic acid sample, binding to the nucleic acid or probe array in a nonspecific manner and providing sites or loci for the non-specific binding of labeled nucleic acid sample. Also, labeled nucleic acid samples may bind non-specifically to nucleic acid arrays for example via electrostatic interactions.

In accordance with one aspect of the present invention, it has been determined that background signal may interfere with or render less interpretable signal generated by specific binding to the probe array of labeled nucleic acid samples. It has also been determined in context with one aspect of the present invention that background un-desirably lowers the signal to noise ratio (SNR) and generates an overall loss of sensitivity for nucleic acid arrays.

Methods are also presented for hybridizing nucleic acid arrays with nucleic acid samples comprising incubating the array with the sample in the presence of a PAP. Hybridization buffers comprising a PAP are also presented.

Preferably, the nucleic acid array is a DNA microarray. More preferably, the nucleic acid array is an oligonucleotide array. The nucleic acid sample is preferably RNA. More preferably, the sample is cRNA. Most preferably the sample is cRNA comprising one or more nucleotides having biotin labels.

PAP are preferably selected from the group consisting of water soluble poly-phosphate or poly-sulfate derivatives of polymers bearing pendant hydroxyl groups, water soluble poly-phosphate or poly-sulfonate of poly-saccharides, and poly(hydroxyalkyl phosphate or phosphonate) polymers. PAPs are also preferably poly-vinyl phosphate, poly-acrylic acid and poly-maleic acid.

### BRIEF DESCRIPTION OF THE DRAWINGS

FIG. 1 shows the effect of PAPs on background of standard samples.

FIG. 2 shows the effect of PAPs on absolute calls of standard samples.

FIG. 3 shows the effect of PAPs on average signal of standard samples.

FIG. 4 shows array images of PAP-treated normal background samples.

FIG. 5 shows the effect of PAPs on background of rat high-background samples.

FIG. 6 shows the effect of PAPs on noise of rat high-background samples.

FIG. 7 shows the effect of PAPs on present calls of rat high-background samples.

FIG. 8 shows the effect of PAPs on spike sensitivity of rat high-background samples.

FIG. 9 shows the effect of PAPs on signal of rat high-background samples.

FIG. 10 shows the effect of PAPs on scaling of rat high-background samples.

FIG. 11 shows array images of PAP-treated rat high-background samples.

FIG. 12 shows array images of PAP-treated artificial high-background samples.

FIG. 13 shows the effect of PAPs on background of artificial high-background samples.

FIG. 14 shows the effect of PAPs on absolute calls of artificial high-background samples.

FIG. 15 shows the effect of PAPs on average signal of artificial high-background samples.

FIG. 16 shows the effect of PVPS on background of artificial high-background samples.

FIG. 17 shows the effect of PVPS on present calls of artificial high-background samples.

FIG. 18 shows the effect of PVPS on average signal of artificial high-background samples.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. No. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

One of skill in the art will appreciate that in order to measure the transcription level (and thereby the expression level) of a gene or genes, it is desirable to provide a nucleic acid sample comprising mRNA transcript(s) of the gene or genes, or nucleic acids derived from the mRNA transcript(s). As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, suitable samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

In a particularly preferred embodiment, where it is desired to quantify the transcription level (and thereby expression) of a one or more genes in a sample, the nucleic acid sample is one in which the concentration of the mRNA transcript(s) of the gene or genes, or the concentration of the nucleic acids derived from the mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes. Where more precise quantification is required appropriate controls can be run to correct for variations introduced in sample preparation and hybridization as described herein. In addition, serial dilutions of “standard” target mRNAs can be used to prepare calibration curves according to methods well known to those of skill in the art. Of course, where simple detection of the presence or absence of a transcript is desired, no elaborate control or calibration is required.

In the simplest embodiment, such a nucleic acid sample is the total mRNA isolated from a biological sample. The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

The nucleic acid (either genomic DNA or mRNA) may be isolated from the sample according to any of a number of methods well known to those of skill in the art. One of skill will appreciate that where alterations in the copy number of a gene are to be detected genomic DNA is preferably isolated. Conversely, where expression levels of a gene or genes are to be detected, preferably RNA (mRNA) is isolated.

Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization with Nucleic Acid Probes, Part I. Theory and Nucleic Acid Preparation, P. Tijssen, ed. Elsevier, N.Y. (1993)).

In a preferred embodiment, the total nucleic acid is isolated from a given sample using, for example, an acid guanidinium-phenol-chloroform extraction method and polyA.sup.+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of radioactivity (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al., Academic Press, Inc. N.Y., (1990).

Other suitable amplification methods include, but are not limited to polymerase chain reaction (PCR) (Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

In a particularly preferred embodiment, the sample mRNA is reverse transcribed with a reverse transcriptase and a promoter consisting of oligo dT and a sequence encoding the phage T7 promoter to provide single stranded DNA template. The second DNA strand is polymerized using a DNA polymerase. After synthesis of double-stranded cDNA, T7 RNA polymerase is added and cRNA is transcribed from the cDNA template. Successive rounds of transcription from each single cDNA template results in amplified RNA. Methods of in vitro polymerization are well known to those of skill in the art (see, e.g., Sambrook, supra.) and this particular method is described in detail by Van Gelder, et al., Proc. Natl. Acad. Sci. USA, 87: 1663-1667 (1990) who demonstrate that in vitro amplification according to this method preserves the relative frequencies of the various RNA transcripts. Moreover, Eberwine et al. Proc. Natl. Acad. Sci. USA, 89: 3010-3014 provide a protocol that uses two rounds of amplification via in vitro transcription to achieve greater than 106 fold amplification of the original starting material thereby permitting expression monitoring even where biological samples are limited.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The protocols cited above include methods of generating pools of either sense or antisense nucleic acids. Indeed, one approach can be used to generate either sense or antisense nucleic acids as desired. For example, the cDNA can be directionally cloned into a vector (e.g., Stratagene's p Bluscript II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lamda vectors designed for Cre-loxP plasmid subcloning (see e.g., Palazzolo et al., Gene, 88: 25-36 (1990)).

In a particularly preferred embodiment, a high activity RNA polymerase (e.g. about 2500 units/.mu.L for T7, available from Epicentre Technologies) is used.

Nucleic Acid Labeling

In a preferred embodiment, the hybridized nucleic acids are detected by detecting one or more labels attached to the sample nucleic acids. The labels may be incorporated by any of a number of means well known to those of skill in the art. However, in a preferred embodiment, the label is simultaneously incorporated during the amplification step in the preparation of the sample nucleic acids. For example, polymerase chain reaction (PCR) with labeled primers or labeled nucleotides will provide a labeled amplification product. The nucleic acid (e.g., DNA) is be amplified in the presence of labeled deoxynucleotide triphosphates (dNTPs). The amplified nucleic acid can be fragmented, exposed to an oligonucleotide array, and the extent of hybridization determined by the amount of label now associated with the array. In a preferred embodiment, transcription amplification, as described above, using a labeled nucleotide (e.g. fluorescein-labeled UTP and/or CTP) incorporates a label into the transcribed nucleic acids.

Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., .sup.3H, .sup.125 I, .sup.35 S, .sup.14 C, or .sup.32 P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an aviden-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are preferred and easily added during an in vitro transcription reaction. In a preferred embodiment, fluorescein labeled UTP and CTP are incorporated into the RNA produced in an in vitro transcription reaction as described above. cRNA, according to the present invention, is preferably labeled with biotin.

Also provided according to the present invention is a method for detecting hybridization of a nucleic acid sample to a nucleic acid array. This method has the following steps: providing a nucleic acid sample comprising mRNA transcripts of one or more genes; reverse transcribing the nucleic acid sample with a reverse transcriptase and a promoter consisting of oligo dT and a sequence encoding the phage T7 promoter to provide single stranded DNA template; synthesizing double stranded cDNA from the single stranded DNA template using DNA polymerase to provide cDNA template; transcribing the cDNA template with T7 RNA polymerase to provide cRNA; fragmenting the cRNA with an RNase to provide fragmented cRNA; and hybridizing said fragmented cRNA to a nucleic acid array. According to the present invention, the preceding method also preferably includes an additional step of end labeling the fragmented cRNA. Preferably, the end labeling is with biotin.

Also, according to the present invention, the step of transcribing the cDNA template may preferably be carried out in the presence of biotin labeled ribonucleotides to provide biotin labeled cRNA. Preferred embodiments with respect to the RNase enzyme and fragment size are as set forth above.

A nucleic acid array according to the present invention is any solid support having a plurality of different nucleotide sequences attached thereto or associated therewith. One preferred type of nucleic acid array that is useful in the present invention include those that are commercially available from Affymetrix (Santa Clara, Calif.) under the brand name GeneChip®. Example arrays are shown on the website at affymetrix.com.

GeneChip Analysis.

GeneChip® nucleic acid probe arrays are manufactured using technology that combines photolithographic methods and combinatorial chemistry. In a preferred embodiment, over 280,000 different oligonucleotide probes are synthesized in a 1.28 cm×1.28 cm area on each array. Each probe type is located in a specific area on the probe array called a probe cell. Measuring approximately 24 μm×24 μm, each probe cell contains more than 10⁷ copies of a given oligonucleotide probe.

Probe arrays are manufactured in a series of cycles. A glass substrate is coated with linkers containing photolabile protecting groups. Then, a mask is applied that exposes selected portions of the probe array to ultraviolet light. Illumination removes the photolabile protecting groups enabling selective nucleotide phosphoramidite addition only at the previously exposed sites. Next, a different mask is applied and the cycle of illumination and chemical coupling is performed again. By repeating this cycle, a specific set of oligonucleotide probes is synthesized, with each probe type in a known physical location. The completed probe arrays are packaged into cartridges.

During the laboratory procedure, biotin-labeled RNA fragments referred to as the RNA target are hybridized to the probe array. The hybridized probe array is stained with streptavidin phycoerythrin conjugate and scanned by the Hewlett-Packard (HP) GeneArray™ Scanner at the excitation wavelength of 488 nm. The amount of emitted light at 570 nm and above is proportional to the amount of bound labeled target at each location on the probe array.

Step 1: Target Preparation

A total RNA population may be isolated from tissue or cells and reverse transcribed to produce cDNA. Then, in vitro transcription (IVT) produces biotin-labeled cRNA from the cDNA. The cRNA may be fragmented before hybridization.

Step 2: Target Hybridization

After the biotin-labeled cRNA is fragmented, a hybridization buffer is prepared, which includes labeled sample (0.05 μg/μl), probe array controls (1.5, 5, 25 and 100 pM respectively), herring sperm DNA (0.1 mg/ml), and BSA (0.5 mg/ml). A cleanup procedure is performed on the hybridization buffer after which 200 μl is applied to the probe array through one of the septa in the array. It is then hybridized to the probes on the probe array during a 16-hour incubation at 45° C.

The hybridization protocol involves the following: (1) equilibrate probe array to room temperature immediately before use; (2) heat the sample(s) to 95° C. for 5 minutes in a heat block; (3) meanwhile, wet the array by filling it through one of the septa with 1× Hybridization Buffer (1M NaCl, 0.1 M MES pH 6.7, 0.01% Triton X-100) using a micropipettor and appropriate tips; incubate the probe array at the hybridization temperature for 10 minutes with rotation; (5) after incubation at 95° C. (step #2 above), transfer the samples to a 45° C. heat block for 5 minutes; (5) spin samples at maximum speed in a microcentrifuge for 5 minutes to remove any insoluble material from the hybridization mixture; (6) remove the buffer solution from the probe array cartridge and fill with 200 μl of the clarified hybridization buffer avoiding any insoluble matter in the 20 μl at the bottom of the tube; (7) place probe array in rotisserie box in 45° C. oven; load probe arrays in a balanced configuration around rotisserie axis; rotate at 60 rpm; and (8) hybridize for 16 to 40 hours.

Step 3: Probe Array Washing, Staining, and Fluidics Station Setup

Immediately following the hybridization, the hybridized probe array undergoes manual washing and staining, then washing on the fluidics station. The protocol involves the following: (1) remove the hybridization buffer from the probe array and set it aside in a microcentrifuge tube; store on ice during the procedure or at −20° C. for long-term storage; (2) rinse the probe array by pipetting 200 μl 1×MES buffer pH 6.7 through one of the probe array septa; (3) fill the probe array septa with 200 μl 6×SSPE-T (300 ml of 20×SSPE and 500 μl of 10% Triton X 100 to 700 ml of water, final pH 7.6) and wash with 6×SSPE-T on the fluidics station with wash A cycle (10 cycles, drain and fill twice each cycle); (4) remove the 6×SSPE-T and rinse the probe array with 0.1×MES buffer pH 6.7 (0.1 M MES, 0.1 M NaCl and 0.01% Triton); (5) fill the probe array with 200 μl 0.1×MES and incubate at 45° C. on the rotisserie at 60 rpm for 30 minutes; and (6) remove the 0.1×MES, rinse the probe array with 1×MES in the probe array while preparing the stain.

Staining the probe array involves preparing Streptavidin Phycoerythrin (SAPE) stain solution. Stain should be stored in the dark and foil wrapped or kept in an amber tube at 4° C. Remove stain from refrigerator and tap the tube to mix well before preparing stain solution. The concentrated stain or diluted SAPE stain solution should not be frozen. The SAPE stain should be prepared immediately before use.

For each probe array to be stained, combine the following components to a total volume of 200 μl (1:100 dilution of SAPE, final concentration of 10 μg/ml): 188 μl 1×MES; 10 μl of 50 mg/ml acetylated BSA (final concentration of 2.5 mg/ml); and 2 μl of 1 mg/ml streptavidin phycoerythrin (SAPE).

Remove the 1×MES and apply the stain solution to the probe array. Incubate for 15 minutes at 60 rpm at room temperature or 40° C.

Remove the stain and fill the probe array with 6×SSPE-T. Wash the probe array with 6×SSPE-T on the fluidics station with wash A cycle.

The experiment parameters are preferably defined using commercially available GeneChip® software (Affymetrix, Santa Clara, Calif.) on a PC-compatible workstation with a Windows NT® operating system. The probe array type, sample description, and comments are entered in the software and saved with a unique experiment name.

The user protocol involves the following: (1) launch the software from the workstation and choose Experiment Info from the Run menu; alternatively, click the New Experiment icon on the GeneChip® software tool bar; the Experiment Information dialog box will appear allowing the experiment name to be defined along with several other parameters such as probe array type, sample description, and comments; (2) type in the experiment name; click on the box to the right of Probe Array type and select the probe array type from the drop-down list; experiment name and probe array type are required; complete as much of the other information as desired; the protocol information at the bottom of the dialog box will be imported to the experiment information dialog box after the hybridization and scan have been completed; (3) save the experiment by choosing Save; the name of the experiment will be used by the software to access the probe array type and data for the sample while it is being processed; data files generated for the sample will be automatically labeled to correspond to the experiment name; the Protocol section of the dialog box will be filled in by the software; and (4) close the Experiment Information dialog box.

The GeneChip® Fluidics Station 400 is preferably used to wash the probe arrays. It is operated using the GeneChip® software as follows: (1) choose Fluidics from the Run menu; alternatively, click the Start Protocol icon on the GeneChip® software tool bar; the Fluidics Station dialog box will appear with a drop-down list for the experiment name; a second list is accessed for the Protocol for each of the four fluidics station modules; (2) prime the fluidics station, by clicking Protocol in the Fluidics Station dialog box; choose Prime for the respective modules in the Protocol drop-down list; change the intake buffer reservoir A and B to 6×SSPE-T; click Run for each module to begin priming; priming should be done whenever the fluidics station is first started up, when wash solutions are changed, after washing if a shutdown has been performed on any module, and if the LCD window instructs the user to prime; priming ensures that the wash lines are filled with the appropriate buffer and the fluidics station is ready for washing; a prime takes approximately 3 to 5 minutes to complete; the fluidics station LCD window and the Fluidics Station dialog box will display the status of the prime and give instructions as it progresses; follow the instructions on the LCD window and dialog box; when priming is complete, the LCD window and dialog box will indicate that the fluidics station is ready to run a wash; (3) wash the probe array on the fluidics station, by customizing the HYBWASH protocol to create a wash of 10 cycles with 2 mixes per cycle with 6×SSPE-T at room temperature; in the Fluidics Station dialog box on the workstation, select the correct experiment name in the drop-down Experiment list; the probe array type will appear automatically; in the Protocol drop-down list, select the modified HYBWASH protocol created in step 1 to control the wash of the probe array; if a customized protocol is run, check the parameters of each of the protocols chosen to be sure they are appropriate for your experiment; this can be done in the Fluidics Protocol dialog box found by choosing Edit Protocol under the Tools menu; choose Run in the Fluidics Station dialog box to begin the wash; follow the instructions on the LCD window on the fluidics station; open the probe array holder by pressing down on the probe array lever to the Eject position; place the appropriate probe array into the probe array holder of the selected module and gently push up on the lever to engage it; the latch should be secure when the probe array holder is fully closed; a light click should be heard; engage the probe array holder lever by firmly pushing up on it to the Engage position; the Fluidics Station dialog box and the LCD window will display the status of the wash as it progresses; when the wash is complete, the LCD window will display EJECT CARTRIDGE; eject the probe array by pushing down firmly on the probe array lever; and (4) perform the cleanout procedure, by returning the probe array to the probe array holder; latch the probe array holder by gently pushing it up until a light click is heard; engage by firmly pushing up on the probe array lever to the Engage position; the fluidics station will drain the probe array and then fill it with a fresh volume of the last wash buffer used; when it is finished, if the LCD window displays EJECT CARTRIDGE again, remove the probe array and inspect it again for bubbles; if no bubbles are present, it is ready to scan; after ejecting the probe array from the probe array bolder, the LCD window will display ENGAGE WASHBLOCK; latch the probe array bolder by gently pushing it up and in until a light click is heard; engage the washblock by firmly pushing up on the probe array lever to the Engage position; the fluidics station will automatically perform a Cleanout procedure; the LCD window will indicate the progress of the Cleanout procedure; when the Cleanout procedure is complete, the LCD window should display Washing done, READY; if no other washes are to be performed, place wash lines into a bottle filled with deionized water; choose Shutdown for all modules from the drop-down Protocol list in the Fluidics Station dialog box; click the Run button for all modules; after Shutdown protocol is complete, flip the ON/OFF switch of the fluidics station to the OFF position; and scan the probe array.

Step 4: Probe Array Scan

Once the probe array has been hybridized, stained, and washed, it is scanned. Each workstation running the software can control one scanner. Each scan takes approximately 5 minutes, and two scans are recommended.

The scanner acquires an image of each of the hybridized 24 μm×24 μm probe cells. Each complete probe array image is stored in a separate data file that corresponds to its experiment name and is saved with a data image file (.dat) extension.

The scanner is also controlled by the GeneChip® software. The probe array is scanned after the wash protocols are complete. The probe array scan proceeds as follows: (1) choose Scanner from the Run menu; alternatively, click the Start Scan icon in the GeneChip® software tool bar; the Scanner dialog box will appear with a drop-down list of experiments that have not been run; a scrollable window will also be displayed showing previous scans; choose the experiment name that corresponds to the probe array to be scanned; a previously run experiment can also be chosen from the Previous Experiments list by double-clicking on the name desired; (2) check for the correct pixel value and wavelength of the laser beam; for a 24 μm×24 μm probe array with a phycoerythrin stain: Pixel value=3 μm, Wavelength=570 nm; (3) once the experiment has been selected, click the Start button; a dialog box will prompt the user to load a sample into the scanner; and (4) load the Probe Array into the HP GeneArray™ Scanner; open the sample door on the scanner and insert the probe array into the holder; do not force the probe array into the holder; close the sample door of the scanner; start the Scan, by clicking OK in the Start Scanner dialog box; the scanner will begin scanning the probe array and acquiring data; when Scan in Progress is chosen from the View menu, the probe array image will appear on the screen as the scan progresses.

Step 5: Data Analysis and Interpretation

Data is analyzed using GeneChip® software. In the Image window, a grid is automatically placed over the image of the scanned probe array to demarcate the probe cells. After grid alignment (the user may adjust the alignment if necessary), the mean intensity at each probe cell is calculated by the software. The intensity patterns are analyzed.

After scanning the probe array, the resulting image data created is stored on the hard drive of the GeneChip® workstation as a .dat file with the name of the scanned experiment. In the first step of the analysis, a grid is automatically placed over the .dat file so that it demarcates each probe cell. One of the probe array library files, the .cif file, indicates to the software what size of grid should be used. Confirm the alignment of the grid by zooming in on each of the four corners and the center of the image.

If the grid is not aligned correctly, adjust its alignment by placing the cursor on an outside edge or corner of the grid. The cursor image will change to a small double-headed arrow. The grid can then be moved using the arrow keys or by clicking and dragging its borders with the mouse.

Sample analysis occurs as follows: (1) choose Defaults from the Tools menu to access the Probe Array Call Settings tab dialog box; in the Defaults dialog box, click on the Probe Array Call Settings tab to display probe array calling algorithm choices; (2) highlight GeneChip® Expression and click the Modify button or double click the algorithm name; (3) in the Probe Array Call Settings dialog box, select the probe array type in the drop down list; for that probe array make sure the Use As Current Algorithm cheek box is selected; (4) click the OK button to apply your choices for the selected probe array type; (5) in the Defaults dialog box, click the OK button to apply your choices regarding parameters set by all of the tab dialog boxes in the window; (6) after confirming that the above parameters are correct, select the appropriate image to be analyzed; and (7) select Analysis from the Run menu or click the Run Analysis icon on the GeneChip® software tool bar; the software calculates the average intensity of each probe cell using the intensities of the pixels contained in the cell; pixels on the edges of each cell are not included, which prevents neighboring cell data from affecting a cells calculated average intensity; the calculated average intensity is assigned an X/Y-coordinate position, which corresponds to the cell's position on the array; this data is stored as a .cel file using the same name as the .exp and .dat files; the cel file is an intermediate data file; the software then applies the selected probe array algorithm to determine expression levels for each gene; this is done with reference to the information contained in the .cdf file, the second library file for the probe array; the resulting analysis is automatically displayed as a .chp file in the Expression Analysis window of GeneChip® software; the .chp file has the same name as the .exp, .dat, and cel files.

In accordance with one aspect of the present invention, a method is presented for reducing background signal on a nucleic acid array, the background associated with hybridization of a nucleic acid sample to the array, the method comprising hybridizing the array with the sample in the presence of a poly-anionic polymer (PAP).

In accordance with one aspect of the present invention, without being bound by theory, it has been discovered that background or background signal in the context of nucleic acid arrays hybridized to a sample may be due to a number of factors, including without limitation, impurities in the sample, such as cell debris and salts, which bind to the nucleic acid or probe array in a nonspecific manner and provide sites or loci for the non-specific binding of labeled molecules such as for example biotinylated cRNA samples. It has also been discovered in accordance with one aspect of the present invention that background may be generated via non-specific binding of labeled nucleic acid to a nucleic acid array, such as for example by electrostatic binding.

In the context of the present invention, the term background refers to anything that diminishes or interferes with true signal, generated by specific binding of labeled samples to nucleic acid arrays. Non-specifically bound labeled molecules may provide signal, such as for example fluorescence, which interferes with or renders less interpretable signal such as fluorescence generated by specific binding to the probe array. Background, due to non-specific binding causes a low signal to noise ratio (SNR). High background creates an overall loss of sensitivity in the experiment, so it is desirable to reduce or eliminate background effects during measurement.

Within the context of the present invention, background is synonymous with noise, another term used by those of skill in the art to refer to the generation of signal by virtue of non-specific binding to nucleic acid arrays. Persons of skill in the art understand that background may be assessed both quantitatively and qualitatively.

In accordance with the present invention, it is noteworthy that not all hybridizations of nucleic acid samples to arrays results in background signal which unduly interferes with or renders signal from specifically bound nucleic acids overly difficult to interpret. In the context of GeneChip® Arrays, in a significant majority of experiments, arrays are hybridized to sample and the results may be interpreted with the techniques disclosed above without the need of reducing background signal. In this regard, as described in the examples below, background was purposefully generated in hybridized arrays in order to study the effect of background reducing PAPs. In theory and practice, there is always some non-specific binding of labeled probes to arrays, which generates what may be considered an acceptable level of background. Where background is present, but acceptable, the arrays may be satisfactorily interpreted using controls and software as described above. However, in accordance with the present invention, it has been discovered that certain combinations of arrays and samples give unusually high levels of background signal, causing varying levels of difficulty in interpreting the results (e.g., as discussed below certain samples of rat brain cRNA hybridized to rat arrays). In such cases, it may be desirable to reduce the level of background as described with respect to the instant invention.

According to one aspect of the present invention, it has been discovered that a PAP may be used during hybridization of an array to a nucleic acid sample to reduce background. In accordance with one aspect of the present invention, PAPs are defined in accordance with the present invention as synthetic or natural polymers containing multiple anionic residues or sites. In accordance with the present invention, a PAP is composed of at least two monomers each monomer having the same or different anionic residue of site or residue. In accordance with the present invention the PAP may be a copolymer. In general, in accordance with the present invention, the PAP's are preferably soluble or partially soluble in aqueous environments. However, water solubility is not requisite and solubility issues may be overcome in accordance with the present invention by adjusting the polarity and hyrophobicity/philicity of the hybridization solution or environment. Persons of ordinary skill will recognize that any changes to the hybridization solution must not overly impair the ability of nucleic acids to interact with one another to form duplexes.

In accordance with one aspect of the present invention, without limitation by mechanism, it has been discovered that PAP's may bind to the surface of a nucleic acid array by the same mechanism by which labeled nucleotides and nucleic acids bind non-specifically. If present in sufficient quantities and concentrations in a hybridization buffer, PAPs may compete for and block surface binding sites, thus preventing or reducing non-specific binding of nucleic acid samples, and reducing background signal. The PAPS do not, however, compete with the specific binding of complimentary nucleic acid targets in the sample. Thus, reduction of nonspecific background signal occurs without significant reduction of specific signal generated by appropriate nucleic acid base pairing.

PAPs may be chosen by those of skill in the art according to the disclosures of the present invention. In accordance with one aspect of the present invention, it is preferred that PAPs are selected from the group consisting of water soluble poly-phosphate or poly-sulfate derivatives of natural or synthetic polymers bearing pendant hydroxyl groups, poly-phosphate or poly-sulfate derivatives of poly-saccharides, and poly(hydroxyalkyl phosphate or phosphonate) polymers. PAPs are also preferably selected from the group consisting of poly(hydroxyalkylene phosphates), such as poly(hydroxyethyl phosphate) and poly(hydroxypropyl phosphate) (see, e.g., K. Kajuznynski, et al. (1976) Macromolecules 9, 365); poly-acrylic acids (PAA), poly-maleic acids (PMA), poly-methacrylic acids and poly-vinyl anionic derivatives, preferably poly-vinyl phosphate (PVP) (see, e.g., M. Banks, et al. (1993) Polymer 34, 4547, available from Polysciences, Inc., Warrington Pa.), poly-vinyl sulfate, poly-allyl phosphate, poly-allyl sulfate, poly-vinyl phosphonic acid (PVPS) (available from Clariant GmbH, Wiesbaden, Germany), and poly-vinyl sulfonic acid. Most preferably the poly-anionic polymer is poly-vinyl phosphate.

PAP's are also preferably selected from the group consiting of poly-anionic polypeptides such as, for example, poly-aspartate, poly-glutamate, poly-serine phosphate, and poly-threonine phosphate. PAPs are also preferably poly(hydroxyalkyl phosphate/phosphonate) polymers. PAPs are also preferably selected from poly-anionic polysaccharides, such as for example, glycogen phosphate or sulfate, dextran phosphate (see, e.g., R. A. Whistler, et al. (1969) Arch. Biochem. Biophys. 135, 396) or sulfate and ficoll phosphate or sulfate.

In accordance with one aspect of the present invention, PAPs are any polyphosphorylated form of any polymer bearing pendant hydroxyl groups. Such polymers are preferred as the anionic chain most resembles and therefore will block the non-specific binding of polynucleotide samples and, thus, reduce background. In accordance with one aspect of the present invention, PAPs are preferably prepared and used as aqueous solutions of their Li, Na, or K salts, at a pH of 5-9. The present invention also contemplates that more than one PAP may be used to reduce background from hybridizing a sample to an array. The present invention contemplates that various combinations of different PAPs may be employed within the context of the present invention in a single hybridization buffer.

In accordance with one aspect of the present invention, it is preferred that the nucleic acid array is a DNA microarray. It is particularly preferred that the DNA microarray is an oligonucleotide microarray.

In accordance with one aspect of the present invention, it is preferred the sample is RNA. It is also preferred in accordance with the present invention that the sample is cRNA. In a particularly preferred embodiment of one aspect of the present invention, the cRNA is composed of chains of nucleotides having one or more biotin labels.

In yet another preferred embodiment of the present invention, the sample is DNA. In a particularly preferred embodiment, the sample is DNA labeled with biotin.

In accordance with one aspect of the present invention, a hybridization buffer for hybridizing a nucleic acid sample to a nucleic acid array is presented, said buffer comprising a poly-anionic polymer (PAP), which are described for purposes of the present invention above. Preferably, the hybridization buffer contains the PAP in an amount between 1 to 100 mM. More preferably, the hybridization buffer contains the PAP in an amount between 5 to 50 mM. Most preferably, the PAP is present in the hybridization buffer in an amount between 5-10 mM.

In one aspect of the present invention, a method is presented for hybridizing a nucleic acid sample to a nucleic acid array, said method comprising the step of incubating said sample to said array in the presence of a polyanionic polymer. The PAP, array and sample are preferably as set forth above. In a particularly preferred embodiment of the present invention, the PAP is poly-vinyl phosphate, present at 6 mM, the array is an oligonucleotide array, and the sample is cRNA comprising one or more nucleotides labeled with biotin.

### EXAMPLES

Four polyanionie polymers (PAPs) were tested for their ability to reduce background on DNA microarrays: poly-vinyl phosphate (PVP), poly-vinyl phosphonic acid (PVPS), poly-maleic acid (PMA) and poly-acrylic acid (PAA). PAA was obtained from Polysciences, Inc. in the ˜3000 molecular weight. PVP, PMA and PAA reduced background by 50% using rat cRNA hybridized to medium-background rat arrays (GeneChip® Rat Expression Array 230B). This background suppression significantly improved array performance as measured by present calls and spike sensitivity. Artificial high-background target was generated by omitting the final purification step of cRNA synthesis. PMA effectively reduced artificial high-background by 50% resulting in improved array performance. High concentrations of PVPS were also effective at reducing artificial high-background. Because PAPs do not impair the performance of normal-background samples, these polymers may be routinely added to hybridization buffers as a safeguard against high background.

Effect of PAPs on Normal Background Samples

Purpose: Test the effect of polyanionic polymers on normal background samples under standard hybridization conditions.

| | |
|---|---|
| Sample: | Hela cRNA (standard internal labeling) |
| Arrays: | Hg-U133A |
| I. No treatment | 1 chip |
| II. 1:10 dilution of each of 3 | 3 chips |
| PAPs | |
| III. 1:100 dilution of 3 PAPs | 3 chips |

| Polyanionic Polymer Stocks: | [M] | [Na+] | 1:10 | 1:100 |
|---|---|---|---|---|
| PAA = 40% polyacrylic acid = | 4.21 | 4.21 | 0.421* | 0.042 |
| PMA = 50% polymaleic acid = | 3.12 | 6.24 | 0.312* | 0.031 |
| PVP = 10% polyvinyl phosphate = | 0.60 | 0.60 | 0.060 | 0.006 |

*Note: In order to preserve standard hybridization kinetics, NaCl concentration was held constant at 1 M by using lower salt 2X hyb buffer to prepare 1:10 PAA & 1:10 PMA targets.

The effect of three polyanionic polymers (see Table 1) was tested on standard cRNA target prior to testing on high background target. Internally-labeled target cRNA was generated from Hela total RNA following the standard Affymetrix protocol for Eukaryotic expression analysis. In this experiment, we tested the effect of PAA, PMA, and PVP on standard, normal background targets. Each of the PAPs were tested at a 1:10 dilution (i.e., 421 mM PAA, 312 mM PMA, or 60 mM PVP) and a 1:100 dilution (i.e., 42 mM PAA, 31 mM PMA, or 6 mM PVP). An untreated control was included for comparison. The samples were hybridized to Human Genome U133A arrays and processed according to the Affymetrix standard antibody amplification protocol.

TABLE 1
Polyanionic polymers tested for background reduction

| Anionic Polymers: | Concentration | Structure |
|---|---|---|
| polyvinyl phosphate PVP | 6 mM, 60 mM | 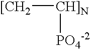 |
| polyacrylic acid (PAA) | 42 mM, 420 mM | 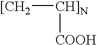 |
| polymaleic acid (PMA) | 30 mM, 300 mM | 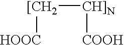 |

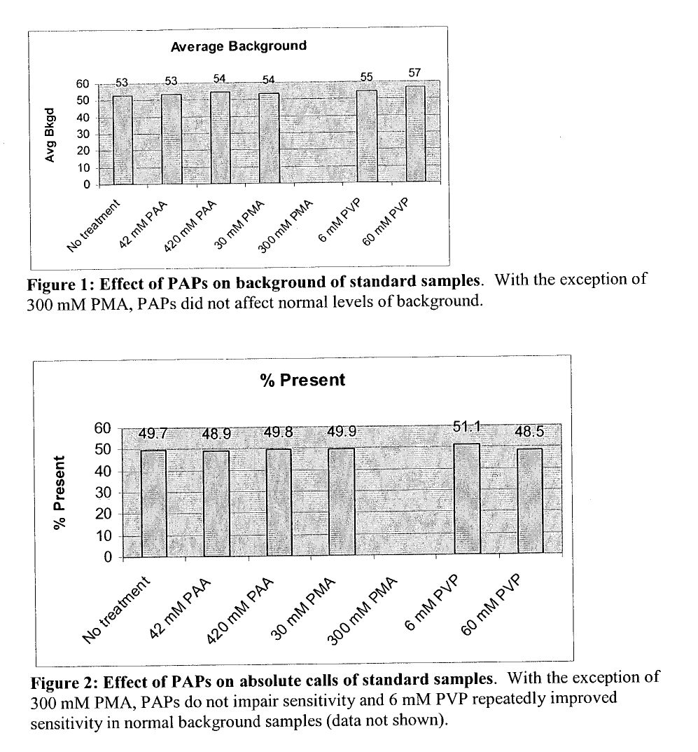

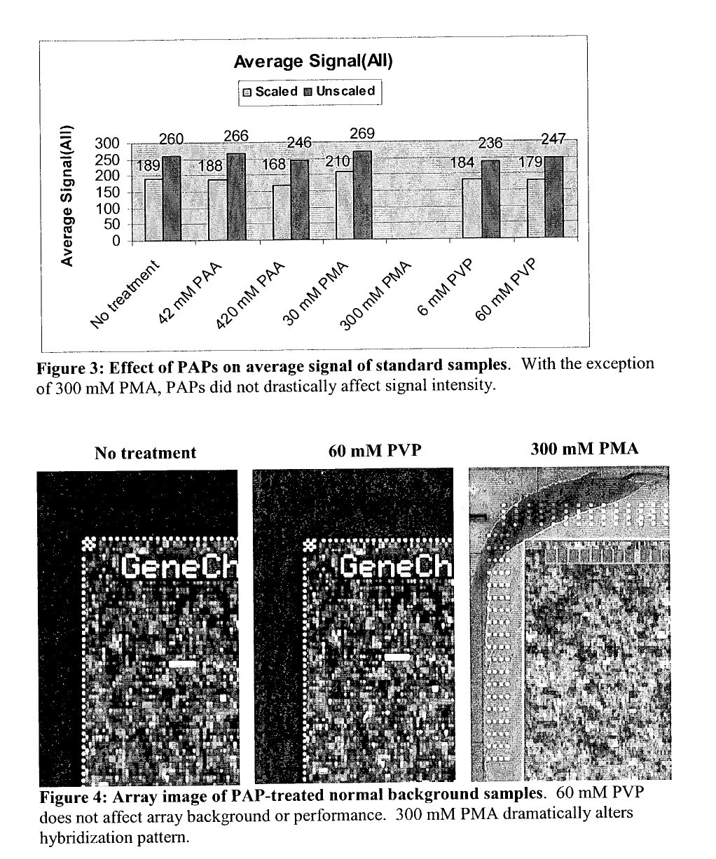

In general, PAPs did not drastically affect the hybridization of normal background target under the conditions tested. As shown in FIGS. 1-3, background, absolute calls and signal intensity of PAP-treated samples are similar to the untreated control. Subtle differences suggest that some PAPs may actually improve standard hybridization characteristics. For example, the 6 mM PVP treatment slightly increased present calls (this was repeated in an independent experiment). The 420 mM PAA, 6 mM PVP, and 60 mM PVP treatments may slightly decrease average signal intensity by approximately 10% (see FIG. 3).

High concentrations of PMA notably impaired array performance. 300 mM PMA strips the Oligo B2 signal that lights up the outside border of the array and changes the hybridization pattern of the sample (see FIG. 4). This effect was repeated in an independent experiment.

Effect of PAPs on Medium-Background Rat Array 230B

Purpose: Test the effect of PAPs on high-background rat samples hybridized to medium-background rat arrays (230 B).

| | |
|---|---|
| Sample: | Rat brain cocktail (previously used targets from Tanimoto |
| | Group, Product Care). |
| Arrays: | Rat Expression Array 230B from lots found to have medium |
| | levels of background. |

| Test Conditions: | # arrays |
|---|---|
| 1) No treatment control | 4* |
| 2) 42 mM PAA (1:100) | 2 |
| 3) 30 mM PMA (1:100) | 2 |
| 4) 6 mM PVP (1:100) | 2 |
| 5) 60 mM PVP (1:10) | 2 |

| Polyanionic Polymer Stocks: | [M] | [Na+] | 1:10 | 1:100 |
|---|---|---|---|---|
| PAA = 40% polyacrylic acid = | 4.21 | 4.21 | 0.421 | 0.042 |
| PMA = 50% polymaleic acid = | 3.12 | 6.24 | 0.312 | 0.031 |
| PVP = 10% polyvinyl phosphate = | 0.60 | 0.60 | 0.060 | 0.006 |

*a volume of water equal to the volume of the PAPs was added to the controls to test for any target concentration effects.

Samples of cRNA from rat brain were obtained which consistently generated high background when hybridized to rat arrays. The following conditions were tested: 1) No treatment (control), 2) 42 mM PAA, 3) 30 mM PMA, 4) 6 mM PVP, 5) 60 mM PVP. There were four replicates of the control and duplicates for each of the PAP conditions tested. The samples were hybridized to “medium background” Rat Expression 230B arrays and processed according to the Affymetrix standard antibody amplification protocol.

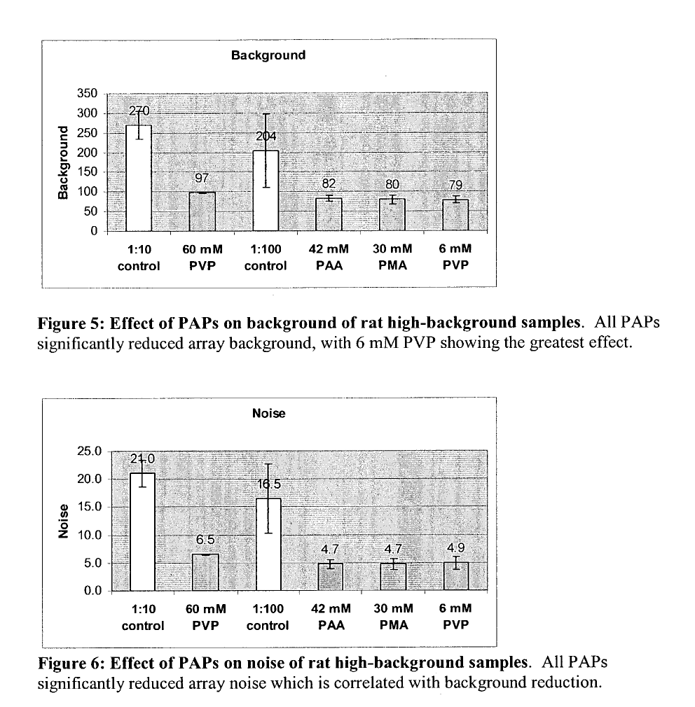

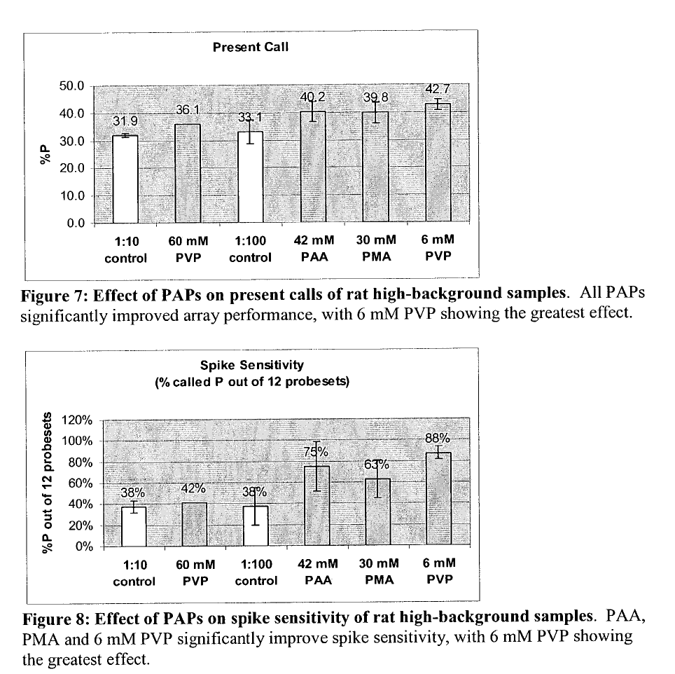

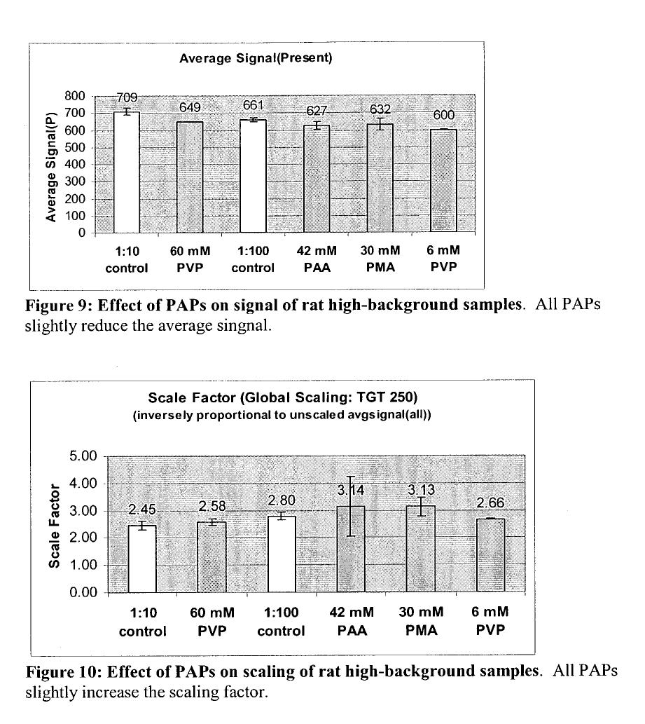

Addition of the PAPs to the high background targets greatly enhanced overall array performance. Hybridization with PAPs reduced background by greater than 50% on average (see FIG. 5); noise was decreased in proportion to the background reduction (see FIG. 6). All of the PAP treatments improved the number of present calls compared to the untreated control (see FIG. 7). The 6 mM PVP treatment had the most significant impact, increasing the present call percentage from 32% in the untreated sample to 43%. The PAPs also improved spike sensitivity. Out of 12 probe sets queried, only 38% were called Present in the untreated, high background sample (see FIG. 8). Again, the 6 mM PVP treated sample was the most sensitive, detecting 88% of the probe sets as Present. The average signal intensity also decreased by approximately 10% (see FIGS. 9 and 10).

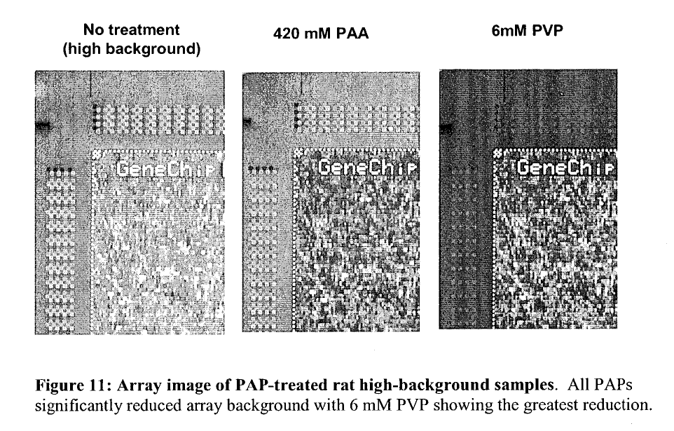

A visual inspection of the array images reveals the dramatic background-reducing effect of PAPs (see FIG. 11). Effect of PAPs on artificial high-background samples

Purpose: Test polyanionic polymers on artificial high-background samples generated by omitting purification or by adding DTT & bio-NTPs to cRNA prior to fragmentation.

| | |
|---|---|
| Sample: | Hela cRNA |
| Array: | HG-U133A |

| Test Conditions: | # arrays |
|---|---|
| 1) Standard (no additional DTT/Bio-NTPs) | 2 |
| 2) No treatment | 2 |
| 3) 42 mM PAA | 2 and 2 unpurified |
| 4) 30 mM PMA | 2 and 2 unpurified |
| 5) 6 mM PVP | 2 and 2 unpurified |

| Polyanionic Polymer Stocks: | [M] | [NaCl] | 1:10 | 1:100 |
|---|---|---|---|---|
| PAA = 40% polyacrylic acid = | 4.21 | 4.21 | 0.421* | 0.042 |
| PMA = 50% polymaleic acid = | 3.12 | 6.24 | 0.312* | 0.031 |
| PVP = 10% polyvinyl phosphate = | 0.60 | 0.60 | 0.060 | 0.006 |

*Note: In order to preserve standard hybridization kinetics, NaCl concentration was held constant at 1 M by using lower salt 2X hyb buffer to prepare 1:10 PAA & 1:10 PMA targets.

High background cRNA was generated from Hela total RNA following the standard Affymetrix protocol for Eukaryotic expression analysis. High background was generated by either omitting the final RNeasy (Qiagen) clean up step or by supplementing additional DTT and labeled ribonucleotides following the RNeasy purification. The amount of DTT and ribonucleotides added was proportional to the amount used in the in vitro transcription reaction to generate the unpurified cRNA. Fragmentation was carried out in the presence of Mg²⁺ and high heat (standard protocol). The following conditions were tested on the artificial high background samples in duplicate: 1) No treatment, 2) 42 mM PAA, 3) 30 mM PMA, 4) 6 mM PVP. For comparison, we prepared a normal background standard in which no additional DTT or labeled ribonucleotides were added to the fragmentation reaction. The samples were hybridized to U133A arrays and processed according to standard protocols.

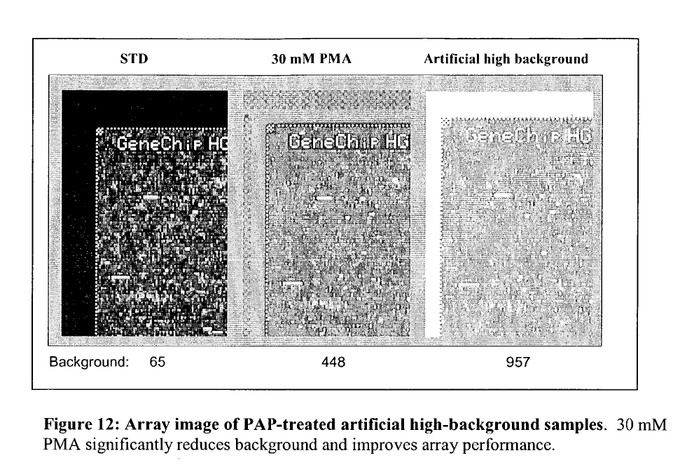

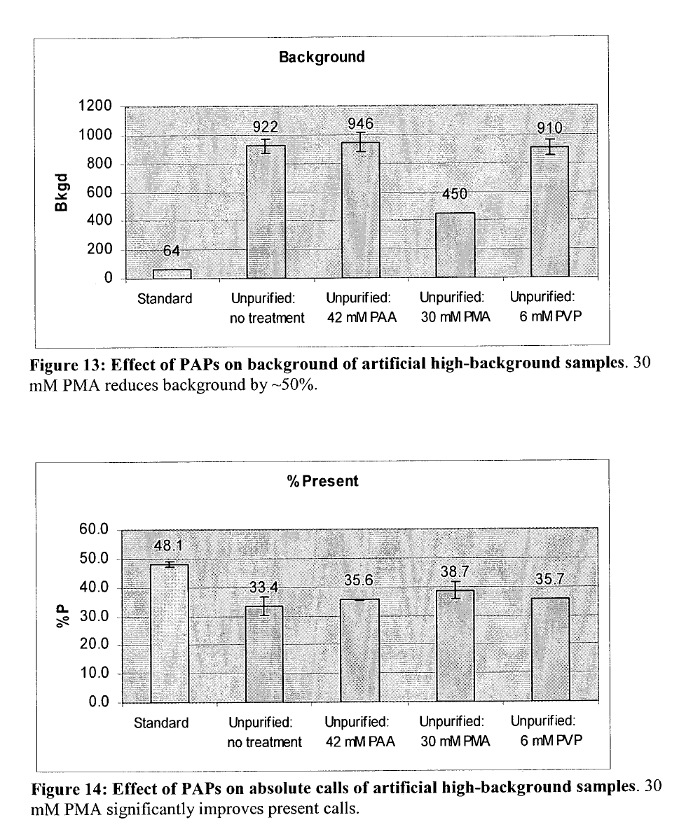

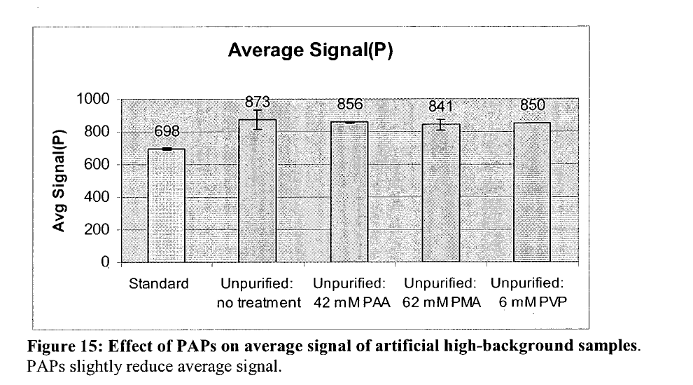

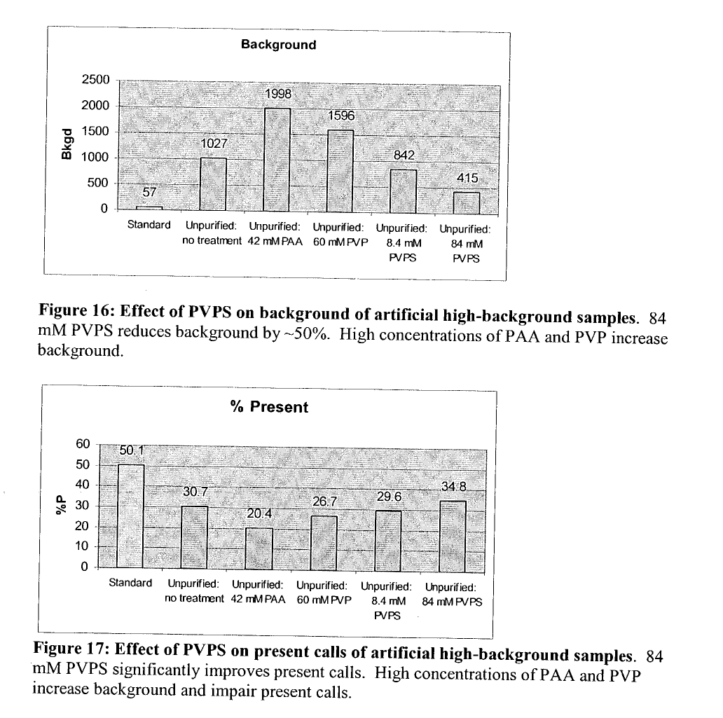

The two methods of generating artificial high background were very comparable and only the unpurified data will be presented. Unpurified, fragmented cRNA produced a background intensity of 922 versus 64 in the normal background control (see FIGS. 12 and 13). The addition of 30 mM PMA reduced background by approximately 50%. The other PAP treatments did not have a significant effect on background at the tested concentrations (see next results section for higher concentrations). The 30 mM PMA treatment and the 6 mM PVP treatment improved the number of present calls from 34% in the untreated sample to approximately 40% (see FIG. 14). This was still much lower than the standard, normal background sample (48% P). Average signal is slightly reduced by the addition of PAPs (see FIG. 15).

Effect of High PAP Concentration and PVPS on Artificial High-Background

Purpose: Test effect of poly vinylphosphonic acid (PVPS) & higher concentrations of PAA & PVP on artificial high-background (unpurified) cRNA samples.

| | |
|---|---|
| Sample: | Hela cRNA |
| Array: | HG-U133A |

| Test Conditions: | # arrays |
|---|---|
| 1) Standard (purified cRNA) | 1 |
| 2) No treatment | 1 |
| 3) 421 mM PAA | 1 |
| 4) 60 mM PVP | 1 |
| 5) 8 mM PVPS | 1 |
| 6) 84 mM PVPS | 1 |

| Polyanionic Polymer Stocks: | [M] | [NaCl] | 1:10 | 1:100 |
|---|---|---|---|---|
| PAA = 40% polyacrylic acid = | 4.21 | 4.21 | 0.421* | 0.042 |
| PMA = 50% poly maleic acid = | 3.12 | 6.24 | 0.312* | 0.031 |
| PVP = 10% poly vinyl phosphate = | 0.60 | 0.60 | 0.060 | 0.006 |
| PVPS = 10% poly vinylphosphonic- | 0.84 | — | 0.084 | 0.008 |
| sodium = | | | | |

In this experiment, we tested the effect of the sodium salt of polyvinlyphosphonic acid (PVPS) on high background resulting from unpurified cRNA. We also tested higher concentrations of PAA and PVP (1:10 dilutions) on these samples. Target cRNA was generated from Hela total RNA following the standard Affymetrix protocol for Eukaryotic expression analysis except the final RNeasy purification step was omitted. The following conditions were tested on the unpurified targets in duplicate: 1) No treatment, 2) 421 mM PAA, 3) 60 mM PVP, 4) 8 mM PVPS, 5) 84 mM PVPS. A purified cRNA target was included as a standard for comparison. The samples were hybridized to U133A arrays and processed according to standard protocols.

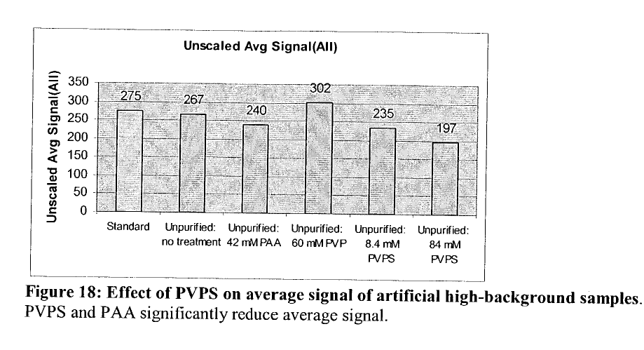

The higher concentrations of PAA and PVP had the unexpected effect of increasing the overall background by 95% and 55%, respectively, compared to the untreated sample (see FIG. 15). The 8 mM PVPS treatment reduced background by approximately 18%, and the 84 mM PVPS treatment decreased background by almost 60%. The present call percentages were inversely proportional to the background intensities (see FIG. 17). Therefore, only the 84 mM PVPS treatment improved the number of present calls over the untreated sample. Compared to previous experiments, 30 mM PMA reduced background more than 84 mM PVPS. The PVPS reduced the average signal (see FIG. 18).

What is claimed is:

1. A method for reducing background signal on a nucleic acid array, said background signal associated with hybridization of said array with a nucleic acid sample, said method comprising hybridizing said array with said sample in the presence of a poly-anionic polymer (PAP).

2. A method according to claim 1 wherein said nucleic acid array is a DNA microarray.

3. A method according to claim 2 wherein said DNA microarray is an oligonucleotide microarray.

4. A method according to claim 1 wherein said sample comprises RNA.

5. A method according to claim 1 wherein said sample comprises cRNA.

6. A method according to claim 1 wherein said sample comprises cRNA comprising one or more nucleotides having biotin labels.

7. A method according to claim 1 wherein said sample comprises DNA.

8. A method according to claim 7 wherein said DNA is labeled with biotin.

9. A method according to claim 1 wherein said PAP is selected from the group consisting of water soluble poly-phosphate or poly-sulfate derivatives of polymers bearing pendant hydroxyl groups and poly(hydroxyalkyl phosphate or phosphonate) polymers.

10. A method according to claim 1 wherein said PAP is poly-vinyl phosphate.

11. A method according to claim 1 wherein said PAP is poly-vinyl phosphonate.

12. A method according to claim 1 wherein said PAP is poly-acrylic acid.

13. A method according to claim 1 wherein said PAP is poly-maleic acid.

14. A hybridization buffer for hybridizing a nucleic acid sample to a nucleic acid array, said buffer comprising a poly-anionic polymer.

15. A hybridization buffer according to claim 14 wherein said background reducing reagent is present in said buffer in an amount between 1 to 100 mM.

16. A hybridization buffer according to claim 14 wherein said amount is between 5 to 50 mM.

17. A hybridization buffer according to claim 14 wherein said amount is between 5-10 mM.

18. A hybridization buffer according to claim 14 where said amount is about 6 mM.

19. A method for hybridizing a nucleic acid sample to a nucleic acid array, said method comprising the step of incubating said sample to said array in the presence of a polyanionic polymer.

20. A method according to claim 19 wherein said nucleic acid array is a DNA micro array.

21. A method according to claim 19 wherein said DNA microarray is an oligonucleotide microarray.

22. A method according to claim 19 wherein said sample comprises RNA.

23. A method according to claim 19 wherein said sample comprises cRNA.

24. A method according to claim 19 wherein said sample comprises cRNA comprising one or more nucleotides having biotin labels.

25. A method according to claim 19 wherein said PAP is selected from the group consisting of water soluble poly-phosphate or poly-sulfate derivatives of polymers bearing pendant hydroxyl groups and poly(hydroxyalkyl phosphate or phosphonate) polymers.

26. A method according to claim 19 wherein said PAP is poly-vinyl phosphate.

27. A method according to claim 19 wherein said PAP is poly-vinyl phosphonate.

28. A method according to claim 19 wherein said PAP is poly-acrylic acid.

29. A method according to claim 19 wherein said PAP is poly-maleic acid.

30. A method according to claim 19 wherein said PAP is poly-vinyl phosphate, said array is an oligonucleotide array, and said sample is cRNA comprising one or more nucleotides labeled with biotin.
